(19) United States
(12) Patent Application Publication
(10) Pub. No.: US 2019/0209132 A1
(43) Pub. Date: Jul. 11, 2019

(54) TRANSESOPHAGEAL ULTRASONIC PROBE

(71) Applicant: BEIJING EAST WHALE IMAGE TECHNOLOGY CO., LTD., Beijing (CN)

(72) Inventors: Xun ZHU, Beijing (CN); Junwen XIAO, Beijing (CN); Yi LI, Beijing (CN)

(21) Appl. No.: 15/868,542

(22) Filed: Jan. 11, 2018

(51) Int. Cl. A61B 8/00; A61B 8/12

(52) U.S. Cl. CPC A61B 8/4281; A61B 8/4444; A61B 8/12

(57) ABSTRACT

A transesophageal ultrasonic probe has a probe sound head and a probe tube connecting to the probe sound head. The probe sound head is provided with a chip. The probe sound head is provided with expandable dissolvable materials. When the probe sound head enters the esophagus and reaches the target position, the expandable dissolvable materials expand and drive the probe sound head to move, making the chip on the probe sound head close to the esophagus or the stomach wall. The transesophageal ultrasonic probe can reduce the probe size and diameters of cables, its extracorporeal part is detachable, and the probe can be placed in a patient's body for a long time for real-time ultrasonic image monitoring of the heart.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT Not applicable.

### INCORPORATION-BY-REFERENCE OF MATERIALS SUBMITTED ON A COMPACT DISC

Not applicable.

### BACKGROUND OF THE INVENTION

### 1. Field of the Invention

This invention related to an ultrasonic probe, especially a transesophageal ultrasonic probe.

### 2. Description of Related Art Including Information Disclosed Under 37 CFR 1.97 and 37 CFR 1.98.

Transesophageal echocardiography (TEE) is a disease detection method in which the ultrasonic probe is inserted to the back of the heart through the esophagus to observe lesions inside the heart. TEE is an important method to evaluate the structure and function of the heart and is widely used in diagnosis of cardiovascular diseases, cardiac operations and anesthesiology.

Compared with trans-thoracic echocardiography (TTE), TEE has obvious advantages. In clinical application, conventional TTE is disturbed by the chest wall and lungs, especially in patients with obesity, chronic pulmonary lesions and mechanical ventilation. TEE probes from the back of the heart by placing the ultrasonic probe into the esophagus; close to the heart and the cardiac aorta, the examination is not disturbed by the chest wall and lungs and clear cardiac images can be obtained, which enhances the reliability of cardiovascular disease diagnosis and facilitates the monitoring and evaluation during and after the cardiac operation.

As shown in FIG. 1, the ordinary transesophageal ultrasonic probe comprises the probe sound head 1 and the probe tube 2 connecting to the probe sound head 1, an operating handle 7 connecting to the probe tube 2 and the mechanical control component in the probe tube 2. The mechanical control component can control the chip to move close to the esophagus or the stomach wall.

For some patients, continuous cardiac image monitoring is needed and may even last for 72 hours. For the ordinary transesophageal probe, since the mechanical control component is disposed in the probe tube 2, the probe tube 2 has a large diameter and it's not suitable to place it into the patient's body for a long time.

### BRIEF SUMMARY OF THE INVENTION

The purpose of this invention is to provide a transesophageal ultrasonic probe which allows the probe to be placed in the patient's body for a long time.

To achieve the above purpose, this invention provides a transesophageal ultrasonic probe. The transesophageal ultrasonic probe comprises a probe sound head and a probe tube connecting to the probe sound head; the probe sound head is provided with a chip; the probe sound head is provided with expandable dissolvable materials; when the probe sound head enters the esophagus and reaches the target position, the expandable dissolvable materials expand and drive the probe sound head to move, making the chip on the probe sound head close to the esophagus or the stomach wall.

Preferably, the expandable dissolvable materials are highly expandable dissolvable chitosan high polymer materials.

Preferably, the expandable dissolvable materials are placed on the probe sound head through a materials container.

Preferably, the materials container adopts dissolvable materials that can dissolve in the digestive tract.

Preferably, the materials container has holes through which liquid can enter the materials container.

Preferably, the materials container has a sealable opening.

Preferably, the materials container is made from breakable materials.

Preferably, the materials container and the chip are respectively disposed on both sides of the probe sound head.

Preferably, the materials container is installed on the probe sound head through a connector; when the transesophageal ultrasonic probe is pulled out from the body, the connector breaks the connection between the materials container and the probe sound head.

Preferably, the connector comprises a slot on the probe sound head and a clamping piece fixed on the materials container; the slot and the probe tube and the connecting end of the probe sound head are respectively disposed on opposite ends of the probe sound head; the clamping piece is detachably installed in the slot.

Preferably, the probe tube comprises an intra-corporeal part and an extracorporeal part; the intra-corporeal part and the extracorporeal part are detachably connected.

Preferably, the intra-corporeal part and the extracorporeal part are connected through a connector; the connector comprises a male connector and a female connector.

Preferably, the male connector and the female connector can be locked through a locking device.

The transesophageal ultrasonic probe in this invention is different from existing technology in that the probe sound head is provided with expandable dissolvable materials and when the probe sound head enters the esophagus and reaches the target position, the expandable dissolvable materials expand and drive the probe sound head to move, pressing the chip of the probe sound head onto the esophagus or the stomach wall without mechanical control component which is required in existing technology, thus effectively reducing the diameter of the probe tube; therefore, the transesophageal ultrasonic probe in this invention allows the probe to be placed in the patient's body for a long time.

### BRIEF DESCRIPTION OF THE SEVERAL VIEWS OF THE DRAWINGS

FIG. 1 is the structure diagram of the existing transesophageal ultrasonic probe.

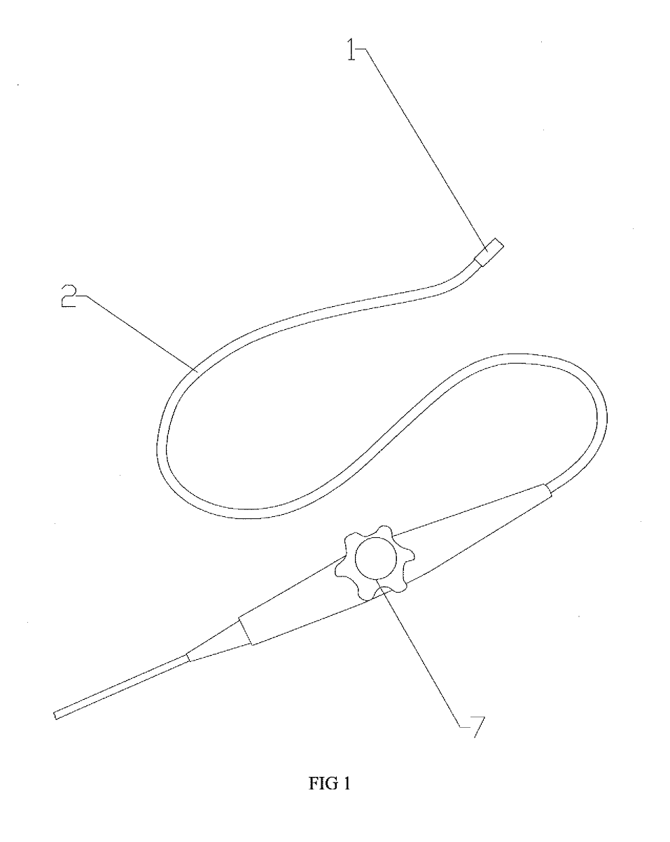

FIG. 2 is the structure diagram of the transesophageal ultrasonic probe in one embodiment of this invention.

FIG. 3 is the structure diagram of expandable dissolvable materials of the transesophageal ultrasonic probe in FIG. 2 after expansion.

FIG. 4 is the structure diagram of the transesophageal ultrasonic probe in FIG. 2 which enters the esophagus.

FIG. 5 is the structure diagram of the materials container with a clamping piece.

FIG. 6 is the structure diagram of the probe sound head with a slot.

FIG. 7 is the structure diagram that shows that the clamping piece in FIG. 5 is installed in the slot in FIG. 6.

FIG. 8 is the structure diagram of the transesophageal ultrasonic probe in another embodiment, wherein the probe tube comprises the intra-corporeal part and the extracorporeal part.

Instruction of drawing reference signs: 1-Probe sound head; 11-Chip; 2-Probe tube; 21-Intra-corporeal part; 22-Extracorporeal part; 3-Expandable dissolvable materials; 4-Materials container; 51-Clamping piece; 52-Slot; 61-Male connector; 62-Female connector; 7-Operating handle.

### DETAILED DESCRIPTION OF THE INVENTION

This invention is further explained in combination with drawings and embodiments. The purpose of the embodiments is only for illustrative purpose and shall not limit the actual scope of protection of this invention in any way and shall not confine the scope of protection of this invention to the embodiments.

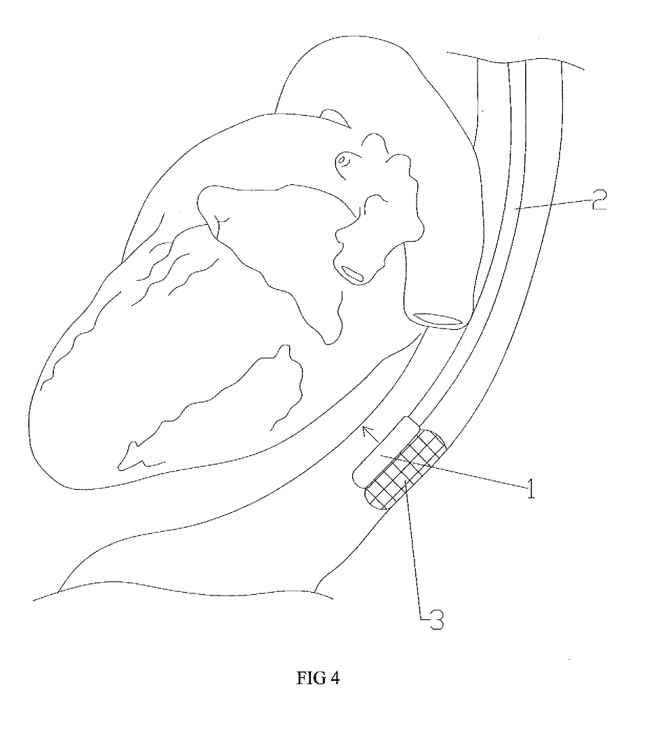

There are some prepositions in this invention. Unless otherwise specified, prepositions such as “on” and “under” are defined in the normal use condition of the transesophageal ultrasonic probe in this invention and mean the same upward and downward directions shown in FIG. 4. “In” and “out” mean the internal and external parts relative to the outlines of components. These prepositions are adopted for the convenience of understanding and shall not limit the scope of protection of this invention.

In this invention, when a component is “fixed” on another component, it can be directly fixed on another component or there can be a connecting component; when a component “connects” to another component, it can directly connect to another component or there can be a connecting component.

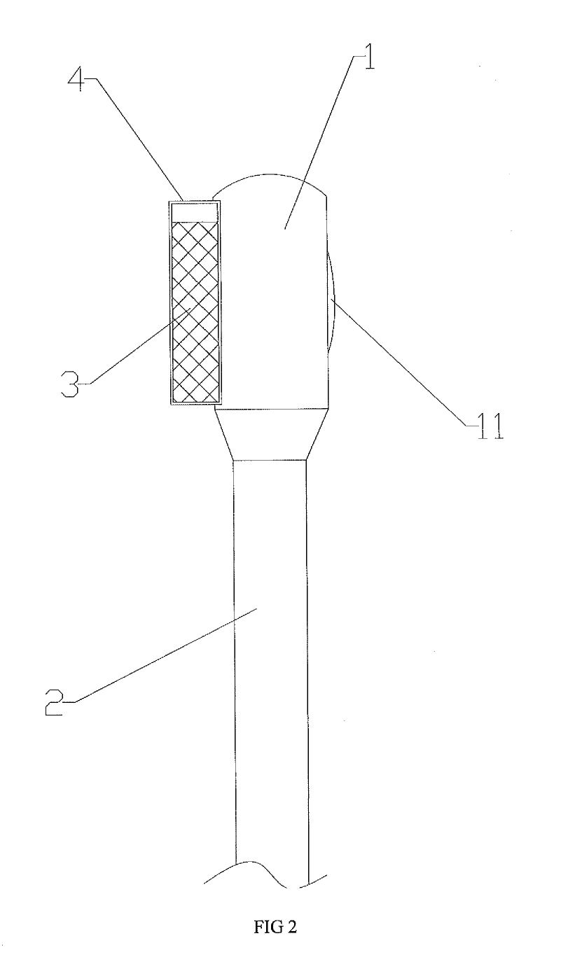

See FIG. 2 as appropriate, the transesophageal ultrasonic probe in the basic embodiment of this invention comprises the probe sound head 1 and the probe tube 2 connecting to the probe sound head 1; the probe sound head 1 is provided with the chip 11; the probe sound head 1 is provided with expandable dissolvable materials 3; when the probe sound head 1 enters the esophagus and reaches the target position, the expandable dissolvable materials 3 expand and drive the probe sound head 1 to move, making the chip 11 on the probe sound head 1 close to the esophagus or the stomach wall.

See FIG. 4, in application of the transesophageal ultrasonic probe in the basic embodiment, the probe sound head 1 is put into the esophagus through incisors and reach the proper position in the esophagus (such as 30 cm inside the esophagus); make the chip 11 on the probe sound head 1 face the part to be examined. FIG. 4 shows that the chip 11 on the probe sound head 1 face the heart; at this moment, the expandable dissolvable materials 3 on the probe sound head 1 absorb digestive juice in the esophagus and expand, driving the probe sound head 1 to move toward the heart and making the chip 11 on the probe sound head 1 close to the esophagus (when the probe sound head is in the stomach, the chip on the probe sound head is close to the stomach wall) to obtain accurate measurement results. In the embodiment, the expandable dissolvable materials 3 are used to replace the mechanical control component to drive the probe sound head 1 to move close to the esophagus or the stomach wall, without using a mechanical control component, thus reducing the diameter of the probe tube and allowing the transesophageal ultrasonic probe in this invention to be placed in the patient's body for a long time.

In this invention, the expandable dissolvable materials 3 can adopt materials that can expand by absorbing digestive juice or gas in the digestive tract and can dissolve slowly in the presence of digestive juice, such as highly expandable dissolvable chitosan high polymer materials, sodium polycrylate resin, starch super absorbent resin, fiber super absorbent resin and natto resin.

In this invention, the expandable dissolvable materials 3 preferably adopt highly expandable dissolvable chitosan high polymer materials. Highly expandable dissolvable chitosan high polymer materials are composed of carboxymethyl cellulose, starch and chitosan. The materials are similar to sponge in structure; it is spongy after expansion and won't pose too much pressure on the esophagus and can slowly dissolve in the presence of digestive juice. Expandable dissolvable chitosan high polymer materials are dissolvable materials with good biocompatibility.

In this invention, the expandable dissolvable materials 3 can be placed on the probe sound head 1 in any proper way. For example, making a holding slot on the probe sound head 1; putting the expandable dissolvable materials in the holding slot through the opening of the holding slot; and sealing the opening of the holding slot with a film that can dissolve in the presence of digestive juice (such as films made of water-soluble edible polymers, including Pulullan film or starch film). The thickness of the film is determined according to the time taken by the probe sound head 1 to reach the target position via the esophagus. When the probe sound head 1 reaches the target position, the film that seals the opening of the holding slot is dissolved in the presence of digestive juice; the expandable dissolvable materials 3 in the holding slot expand by absorbing digestive juice, driving the probe sound head 1 to a proper position.

See FIG. 2, in a preferred embodiment of this invention, the expandable dissolvable materials 3 are placed on the probe sound head 1 through a materials container 4. The materials container 4 can be in various shapes. FIG. 2 shows a rectangular materials container, but this invention is not limited to this shape.

To allow digestive juice to enter the materials container 4 and contact the expandable dissolvable materials 3, the materials container 4 can adopt dissolvable materials that can dissolve in the digestive tract, such as starch. When the materials container made of starch is dissolved, the expandable dissolvable materials 3 in it can expand by absorbing digestive juice. For a materials container 4 made of starch, its wall thickness can be determined according to the time taken by the probe sound head 1 to reach the target position. For example, the wall on the side away from the probe sound head 1 can have a small thickness, so that the wall on the side can dissolve quickly and the expandable dissolvable materials 3 can expand from the wall to drive the probe sound head 1 to move.

Alternatively, the materials container 4 can have holes through which liquid can enter the materials container 4. When the expandable dissolvable materials 3 in the materials container 4 expand, it bulks up the materials container 4, driving the probe sound head 1 to move.

In this invention, the materials container 4 can adopt breakable degradable materials, such as polylactic acid, dioxanone and magnesium alloy. When the expandable dissolvable materials 3 expand, the materials container 4 is broken and enters the stomach and is digested and dissolved.

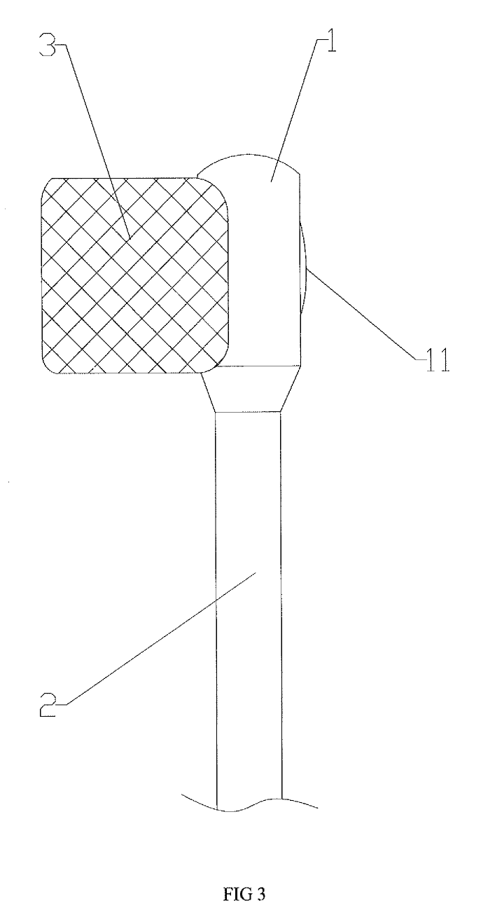

Preferably, the materials container 4 adopts magnesium alloy and has holes. See FIG. 3. When the probe sound head 1 enters the body, moisture in the body can enter the materials container 4 through its holes; when moisture contacts the expandable dissolvable materials, the materials expand and break the thin materials container 4 made of magnesium alloy materials, pressing the probe sound head 1 onto the esophageal wall so that the chip 11 on the probe sound head 1 properly contacts the esophageal wall. The magnesium alloy materials are dissolvable materials with good biocompatibility.

In the above embodiment, preferably, the materials container 4 should dissolve in the digestive tract for no less than 72 hours. The dissolution duration of the materials container 4 can be controlled by controlling the wall thickness of the materials container 4.

In this invention, the materials container 4 can have a sealable opening, so that the materials container 4 can be sealed after the expandable dissolvable materials are placed in the materials container 4.

The materials container 4 can be an elastic container, which means the materials container is made of elastic dissolvable materials, such as silk, polylactic acid fiber, soybean fiber and milk protein fiber. In this case, the materials container 4 has holes though which moisture in the body such as digestive juice can enter the materials container 4; when the moisture contacts the expandable dissolvable materials, the materials expand, deforming the materials container 4 and pressing the probe sound head 1 onto the esophageal wall, so that the chip 11 on the probe sound head 1 properly contacts the esophageal wall.

To allow the chip 11 on the probe sound head 1 to better contact the esophageal wall or the stomach wall, preferably, the materials container 4 and the chip 11 are respectively placed on both sides of the probe sound head 1.

In this invention, the materials container 4 can be fixed on the probe sound head 1 in various ways; for example, making a slot on the probe sound head 1 and clamping the materials container 4 in the slot; alternatively, the materials container 4 can be stuck to the probe sound head by edible glue.

In a preferred embodiment of this invention, the materials container 4 is installed on the probe sound head 1 through a connector. When the transesophageal ultrasonic probe is pulled out from the body, the connector breaks the connection between the materials container 4 and the probe sound head 1. When the detection is finished, the probe sound head 1 can be pulled out of the body, during which the materials container 4 and expandable dissolvable materials produce friction when contacting the esophageal wall, thus breaking the connection; the expandable dissolvable materials and the materials container 4 fall into the stomach and are digested and dissolved.

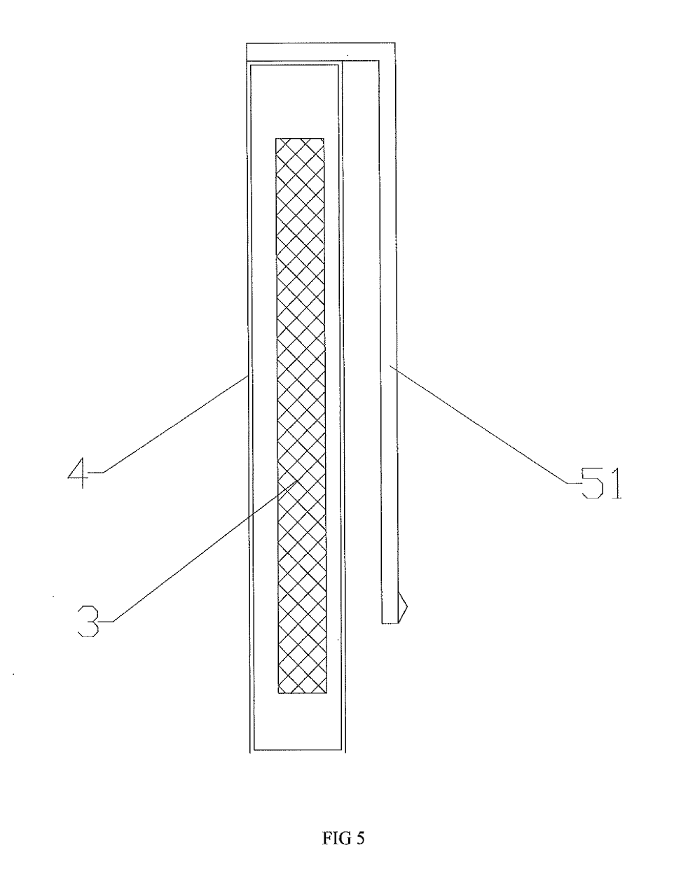

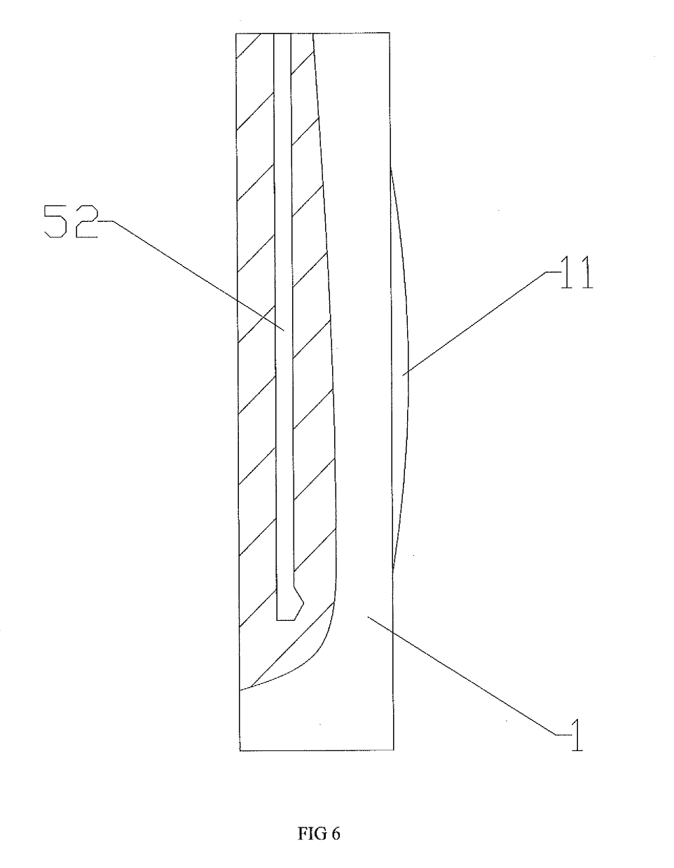

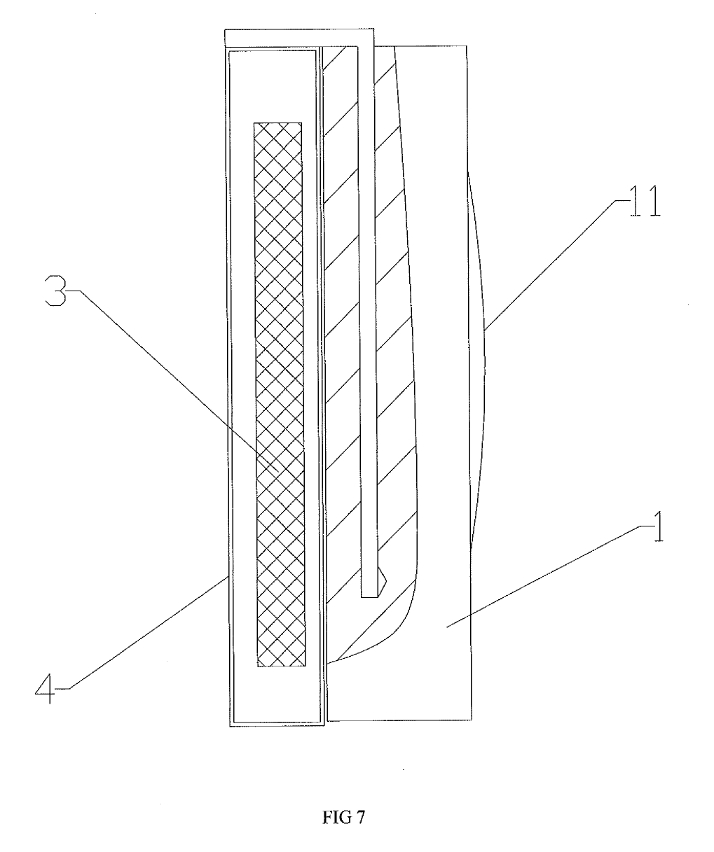

In this invention, the connector can adopt various existing structures. Preferably, as shown in FIGS. 5 to 7, the connector comprises the slot 52 on the probe sound head 1 and the clamping piece 51 fixed on the materials container 4.

The slot 52 and the probe tube 2 and the connecting end of the probe sound head 1 are respectively disposed on opposite ends of the probe sound head 1; that's to say, when the probe sound head 1 is inserted into the esophagus, the slot 52 is at the lower end of the probe sound head 1, so that the materials container 4 won't fall off the probe sound head 1 when the probe sound head 1 is inserted into the esophagus and the materials container 4 can fall off the probe sound head 1 when the probe is pulled out.

The clamping piece 51 is detachably installed in the slot 52. For example, a small bulge can be made on the clamping piece 51 and a small fovea can be made in the corresponding position in the slot 52. When the clamping piece 51 is inserted into the slot 52, the small bulge enters the small fovea. When the probe sound head 1 is pulled out of the body, under the action of small friction, the small bulge separates from the small fovea, thus separating the clamping piece 51 from the slot 52.

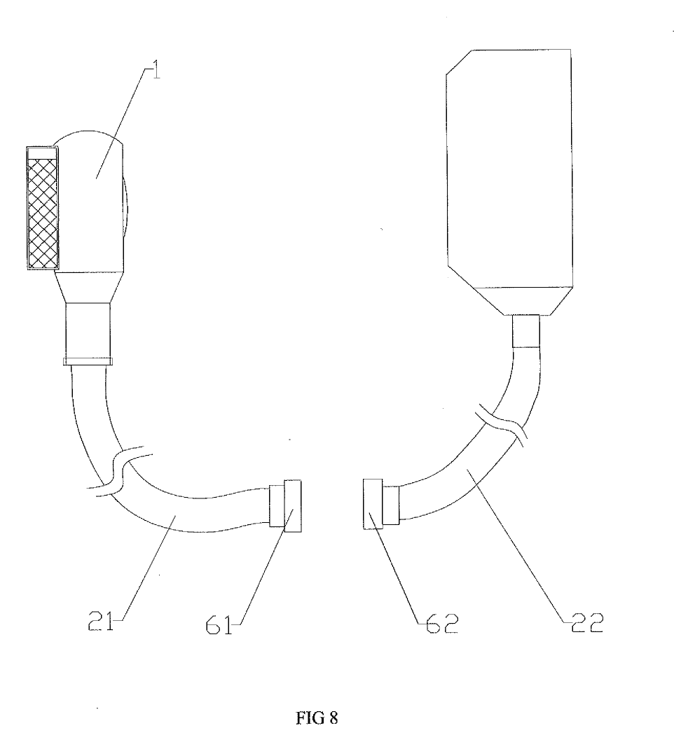

In a more preferred embodiment of this invention, as shown in FIG. 8, the probe tube 2 comprises an intra-corporeal part 21 and an extracorporeal part 22; the intra-corporeal part 21 and the extracorporeal part 22 are detachably connected.

In this way, when the probe sound head 1 is inserted into the body, a transesophageal electrocardiogram can be obtained; when there is no need to observe images, the intra-corporeal part 21 can be separated from the extracorporeal part 22, and the extracorporeal part 22 and the ultrasonic diagnostic apparatus can be removed for other purposes, such as examining other patients. In this case, it's unnecessary to pull out the probe from the patient's body. In case of another examination, connect the intra-corporeal part 21 and the extracorporeal part 22.

The intra-corporeal part 21 and the extracorporeal part 22 can be connected in various ways, for example, via a connector. The connector comprises a male connector 61 and a female connector 62. The male connector 61 and the female connector 62 can be locked through a locking device; the locking device can adopt a mechanical locking structure, such as a buckle, and can be a magnetic locking device.

In this invention, the expandable dissolvable materials 3 are placed on the probe sound head 1; the materials expand in contact with water, pressing the probe sound head 1 onto the esophagus or the esophageal wall and dividing the probe tube 2 into the intra-corporeal part 21 and the extracorporeal part 22; the two parts are separable and the intra-corporeal part 21 is short and light. Therefore, the transesophageal ultrasonic probe in this invention reduces the diameter of the probe tube 2, solves the problem that it's difficult to place the extracorporeal part when the transesophageal ultrasonic probe with a large overall volume and weight is placed in the patient's body for a long time, and makes it possible to keep the transesophageal probe in the patient's body for a long time for examination.

The above embodiments are ordered for the convenience of description only; the sequence shall not represent the quality of embodiments.

Finally, it should be noted that the above embodiments are only used to explain, instead of limiting, the technical solutions of this invention. Although this invention is explained in detail by reference to the above-mentioned embodiments, ordinary technical personnel in this field should understand: they can modify the technical solutions in the above embodiments, or replace some technical features on an equivalent basis; these modifications or replacements shall not separate the essence of corresponding technical solutions from the spirit and scope of the technical solutions of embodiments of this invention.

We claim:

1. A transesophageal ultrasonic probe; the transesophageal ultrasonic probe comprises a probe sound head and a probe tube connecting to the probe sound head; the probe sound head is provided with a chip, characterized in that the probe sound head is provided with expandable dissolvable materials; when the probe sound head enters the esophagus and reaches the target position, the expandable dissolvable materials expand and drive the probe sound head to move, making the chip on the probe sound head close to the esophagus or the stomach wall.

2. The transesophageal ultrasonic probe according to claim 1, characterized in that the expandable dissolvable materials are highly expandable dissolvable chitosan high polymer materials.

3. The transesophageal ultrasonic probe according to claim 1, characterized in that the expandable dissolvable materials is placed on the probe sound head through the materials container.

4. The transesophageal ultrasonic probe according to claim 3, characterized in that the materials container adopts dissolvable materials that can dissolve in the digestive tract.

5. The transesophageal ultrasonic probe according to claim 3, characterized in that the materials container has holes through which liquid can enter the materials container (4).

6. The transesophageal ultrasonic probe according to claim 3, characterized in that the materials container has a sealable opening.

7. The transesophageal ultrasonic probe according to claim 4, characterized in that the materials container is made from breakable materials.

8. The transesophageal ultrasonic probe according to claim 3, characterized in that the materials container and the chip are respectively disposed on both sides of the probe sound head.

9. The transesophageal ultrasonic probe according to claim 3, characterized in that the materials container is installed on the probe sound head through a connector; when the transesophageal ultrasonic probe is pulled out from the body, the connector breaks the connection between the materials container and the probe sound head.

10. The transesophageal ultrasonic probe according to claim 9, characterized in that the connector comprises a slot on the probe sound head and a clamping piece fixed on the materials container; the slot and the probe tube and the connecting end of the probe sound head are respectively disposed on opposite ends of the probe sound head; the clamping piece is detachably installed in the slot.

11. The transesophageal ultrasonic probe according to claim 1, characterized in that the probe tube comprises an intra-corporeal part and an extracorporeal part; the intra-corporeal part and the extracorporeal part are detachably connected.

12. The transesophageal ultrasonic probe according to claim 11, characterized in that the intra-corporeal part and the extracorporeal part are connected through a connector; the connector comprises a male connector and a female connector.

13. The transesophageal ultrasonic probe according to claim 12, characterized in that the male connector and the female connector can be locked through a locking device.
